(19) United States
(12) Patent Application Publication
(10) Pub. No.: US 2025/0372219 A1
(43) Pub. Date: Dec. 4, 2025

(54) METHOD AND APPARATUS FOR AUTOMATED ASSESSMENT OF HOSPITAL QUALITY MEASURES

(71) Applicants: Clairyon, Inc., La Jolla, CA (US); The Regents of the University of California, Oakland, CA (US)

(72) Inventors: Aaron BOUSSINA, San Diego, CA (US); Christopher S. JOSEF, San Diego, CA (US); Shamim NEMATI, San Diego, CA (US)

(21) Appl. No.: 19/228,640

(22) Filed: Jun. 4, 2025

Related U.S. Application Data
(60) Provisional application No. 63/656,091, filed on Jun. 4, 2024.

(51) Int. Cl. G16H 10/60; G16H 70/20

(52) U.S. Cl. CPC G16H 10/60; G16H 70/20

(57) ABSTRACT

A hospital quality abstraction is automatically generated from health records. In some examples, a large language model (LLM) is queried with prompts selected to elicit data to generate a hospital quality abstraction report. The LLM outputs are combined with patient data from health records to improve the accuracy of the responses to the LLM queries. These methods and systems may improve the operation of the LLM, which is deeply needed particularly for healthcare.

### CLAIM OF PRIORITY

This patent application claims priority to U.S. Provisional Patent Application No. 63/656,091, titled “METHOD AND APPARATUS FOR AUTOMATED ASSESSMENT OF HOSPITAL QUALITY MEASURES,” and filed on Jun. 4, 2024, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

In 2022, the cost of quality reporting at a single acute care hospital was over $5 million USD and over 100,000 person-hours. Within US physician practices, the annual cost of quality reporting was 785 hours per physician and over $15 billion. Yet despite this massive financial and provider burden, quality measures are often assessed on a small denominator of patients, which limits their statistical validity and leads to delays in both measurement and actions to improve quality.

It would be beneficial to provide techniques and systems for automatically generating quality measurement reports.

### SUMMARY OF THE DISCLOSURE

Described herein are apparatuses, systems, and methods to generate a report associated with hospital quality measures including but not limited to the Centers for Medicare and Medicaid Services (CMS) severe sepsis and septic shock measure (SEP-1), the National Health Safety Network (NHSN) Central Line Associated Blood Stream Infections (CLABSI) measure, and the Agency for Healthcare Research and Quality (AHRQ) Patient Safety Indicator 90 (PSI-90). The report may be generated using a large language model (LLM) where unstructured clinical data is joined with structured data from patient's health records. In some cases, the patient's health records can be electronic health records. In other cases, the patient's health records can be patient reported outcomes or data from data collection devices. A retrieval-augmented generation (RAG) process may be used to enhance LLM responses to inquiries regarding patient treatment by incorporating relevant health record data.

The methods and apparatuses (devices, system, including software, hardware and firmware) described herein may improve the operation of an LLM. Although this is described herein in the specific context of generating specific hospital quality abstraction reports, it should be understood that these techniques may be applied generally to the operation of an LLM. The accuracy of the LLM may be significantly enhanced by using a Retrieval Augmented Generation (RAG) process in combination with the LLM outputs to determine a query response, wherein the RAG process further uses a particular format (e.g., Fast Healthcare Interoperability Resources, FHIR format) and extracts specific resources that are flattened to create nodes within a knowledge graph RAG (KG-RAG), and providing the LLM this KG-RAG to contextualize the LLM's understanding and ability to answer quality abstraction questions. The queries/prompts sent to the LLM may be dynamically generated or modified based on an analysis of historical query responses, user feedback, and/or evolving clinical guidelines. In some cases a second LLM may be used to generate candidate enhanced prompts that are optimized using a Bayesian Optimization sub-routine to select an optimal prompt by maximizing an objective function comprising a match rate with user reported gold-standard labels.

For example, any of the methods described herein can generate a hospital quality abstraction report. Any of the methods may include receiving, by a processor, one or more electronic health records, querying, by the processor, a large language model (LLM) using a Retrieval Augmented Generation (RAG) process to query data from the one or more electronic health records and LLM outputs to determine a query response, and generating, by a processor, a hospital quality abstraction report based on the query response.

For example, described herein are methods (e.g., methods of generating a hospital quality abstraction report) that include: receiving, by a processor, one or more health records; querying, by the processor, one or more prompts to a large language model (LLM) using a Retrieval Augmented Generation (RAG) process to query data from the one or more health records, additional corpora, and LLM outputs to determine a query response, wherein the RAG process further comprises querying electronic health record (EHR) data directly in Fast Healthcare Interoperability Resources (FHIR) format and extracting specific FHIR resources to incorporate structured EHR data as an additional context to answer quality abstraction questions, further wherein an individual FHIR resource is flattened to create nodes within a knowledge graph structure representing an underlying clinical data ontology, and provided to the LLM a knowledge graph RAG (KG-RAG) to contextualize the LLM's understanding and ability to answer quality abstraction questions; dynamically generating or modifying the one or more prompts based on an analysis of historical query responses, user feedback, and/or evolving clinical guidelines; and generating, by a processor, a hospital quality abstraction report based on the query response.

Any of these methods may include using a second LLM to generate candidate enhanced prompts based on updated guidelines and feeding the candidate enhanced prompts and one or more few-shot examples based on user feedback to a Bayesian Optimization sub-routine to select an optimal prompt and a subset of the few-shot examples by maximizing an objective function comprising a match rate with user reported gold-standard labels. For example, the user reported gold-standard labels may comprise answers to quality measure questions.

The querying may include one or more prompts based on guidelines for determining a hospital quality measure assessment from clinical records. The health records may be stored in a predetermined format. The health records may be compliant with a Fast Healthcare Interoperability Resources standard. The querying may be performed within a Health Insurance Portability and Accountability Act (HIPAA) compliant virtual private cloud.

Any of these methods may include determining clinical criteria from the electronic health records, wherein the hospital quality abstraction report is based on the clinical criteria and the query response. For example, the clinical criteria may include any construct that may comprise of one or more clinical findings that are chained together via logical operators (such as AND, OR), such as Systemic Inflammatory Response Syndrome (SIRS), sequential organ failure assessment score (SOFA), Laboratory Confirmed Bloodstream Infection (LCBI) criteria, among others. Any of these methods may include receiving feedback from a user regarding the hospital quality abstraction report; and creating a feedback record based on the received feedback. The querying may include presenting one or more prompts following a chain-of-thoughts prompting strategy. In some examples the querying includes presenting one or more prompts following a few-shot prompting strategy. In some cases the querying includes presenting one or more prompts selected to elicit responses that generate hospital quality assessment report data.

For example, a method may include: receiving, by a processor, one or more health records; querying, by the processor, one or more prompts to a large language model (LLM) using a Retrieval Augmented Generation (RAG) process to query data from the one or more health records, additional corpora, and LLM outputs to determine a query response, wherein the RAG process further comprises querying electronic health record (EHR) data directly in Fast Healthcare Interoperability Resources (FHIR) format and extracting specific FHIR resources to incorporate structured EHR data as an additional context to answer quality abstraction questions, further wherein an individual FHIR resource is flattened to create nodes within a knowledge graph structure representing an underlying clinical data ontology, and provided to the LLM a knowledge graph RAG (KG-RAG) to contextualize the LLM's understanding and ability to answer quality abstraction questions; wherein the one or more prompts is generated using a second LLM to generate candidate prompts based on updated guidelines and feeding the candidate prompts and one or more few-shot examples based on user feedback to a Bayesian Optimization sub-routine to select an optimal prompt and a subset of the few-shot examples by maximizing an objective function comprising a match rate with user reported gold-standard labels, wherein the user reported gold-standard labels comprise answers to quality measure questions; dynamically generating or modifying the one or more prompts based on an analysis of historical query responses, user feedback, and/or evolving clinical guidelines; generating, by a processor, a hospital quality abstraction report based on the query response; and outputting the hospital quality abstraction report.

Also described herein are non-transitory computer-readable storage medium comprising instructions to perform any of these methods. For example, described herein are non-transitory computer-readable storage medium comprising instructions that, when executed by one or more processors of a device, cause the device to perform operations comprising: receiving, by a processor, one or more health records; querying, by the processor, one or more prompts to a large language model (LLM) using a Retrieval Augmented Generation (RAG) process to query data from the one or more health records, additional corpora, and LLM outputs to determine a query response, wherein the RAG process further comprises querying electronic health record (EHR) data directly in Fast Healthcare Interoperability Resources (FHIR) format and extracting specific FHIR resources to incorporate structured EHR data as an additional context to answer quality abstraction questions, further wherein an individual FHIR resource is flattened to create nodes within a knowledge graph structure representing an underlying clinical data ontology, and provided to the LLM a knowledge graph RAG (KG-RAG) to contextualize the LLM's understanding and ability to answer quality abstraction questions; dynamically generating or modifying the one or more prompts based on an analysis of historical query responses, user feedback, and/or evolving clinical guidelines; and output, from the processor, a hospital quality abstraction report based on the query response.

Also described herein are apparatuses configured to perform any of these methods. For example, an apparatus may include: a processor configured to: receive one or more health records; query one or more prompts to a large language model (LLM) using a Retrieval Augmented Generation (RAG) process to query data from the one or more health records, additional corpora, and LLM outputs to determine a query response, wherein the RAG process further comprises querying electronic health record (EHR) data directly in Fast Healthcare Interoperability Resources (FHIR) format and extracting specific FHIR resources to incorporate structured EHR data as an additional context to answer quality abstraction questions, further wherein an individual FHIR resource is flattened to create nodes within a knowledge graph structure representing an underlying clinical data ontology, and provided to the LLM a knowledge graph RAG (KG-RAG) to contextualize the LLM's understanding and ability to answer quality abstraction questions; dynamically generate or modify the one or more prompts based on an analysis of historical query responses, user feedback, and/or evolving clinical guidelines; and generating, by a processor, a hospital quality abstraction report based on the query response.

In any of the methods described herein, the querying may include one or more prompts based on guidelines for determining a hospital quality measure assessment from clinical records. In some cases, the guidelines may be based on those provided by the Centers for Medicare and Medicaid Services.

In any of the methods described herein, the electronic health records may be stored in a predetermined format. In some cases, the electronic health records may be compatible with a Fast Healthcare Interoperability Resources standard. Alternatively, the electronic health records may be stored as unstructured data (e.g., scanned documents).

In any of the methods described herein, the querying of the LLM and/or the generating of the abstraction report may be performed wholly or partially within a virtual private cloud. In some cases operations within the virtual private cloud may be compliant with Health Insurance Portability and Accountability Act standards.

In general, any of the methods may include determining clinical criteria from the one or more electronic health records, where the hospital quality abstraction report is based on the determined clinical criteria and the query response. In some examples, clinical data may include static patient characteristics (height, weight), patient vital-signs, patient comorbidities, patient demographics, patient procedures, patient tests (blood tests, x-rays, vital signs), ordered medications, administered medications, clinical notes, signals from wearable devices, patient-reported audio or text.

Any of the methods described herein may also include receiving feedback from a user regarding a completed hospital quality abstraction report, and creating a feedback record based on the received feedback.

In any of the methods described herein, the querying may include presenting one or more prompts following a chain-of-thoughts prompting strategy. In some other examples, the querying may include presenting one or more prompts following a few-shot prompting strategy, or the querying may include generating multiple LLM outputs and presenting one following a selection strategy.

Any of the methods described herein can include determining patient medication administration information, where the hospital quality abstraction report is further based on the patient medication administration information. Furthermore, any of the methods described herein may include interventions/procedures and/or imaging. Procedures and/or imaging may be included as part of the quality measures described herein.

A non-transitory computer-readable storage is disclosed. The storage medium may include instructions that, when executed by one or more processors or a device, cause the device to perform operations including receiving one or more electronic health records, querying a large language model (LLM) using a Retrieval Augmented Generation (RAG) process to query data from the one or more electronic health records and LLM outputs to determine a query response, and generating a hospital quality abstraction report based on the query response.

An apparatus is disclosed. The apparatus may include a processor configured to receive one or more electronic health records, query a large language model (LLM) using a Retrieval Augmented Generation (RAG) process to query data from the one or more electronic health records and LLM outputs to determine a query response, and generate a hospital quality abstraction report based on the query response.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 illustrates a simplified flow diagram describing a process flow associated with an automatic generation of hospital quality assessment reports, using, for example, sepsis and septic shock quality criteria.

FIG. 2 shows an example simplified block diagram of a report generating system.

FIG. 3 shows a table outlining the demographics of a test cohort.

FIGS. 4A-4D show a table that shows how well a large language model output agrees with the same report prepared by human abstractors.

FIG. 5 shows an example user interface for providing feedback.

FIG. 6 is a flowchart showing an example method 600 for generating a hospital quality assessment report.

FIG. 7 shows a block diagram of a device that may be one example of the report generating system of FIG. 2.

### DETAILED DESCRIPTION

In general, the generation of reports based on the contents of a patient's clinical health records can be time consuming and error prone. In some examples, large volumes of data may need to be reviewed in order to extract relevant report information. Manually abstracting relevant data from a patient's health records and then analyzing this data to determine relevant information may take hours of personnel time costing a great deal of money.

The present disclosure is related to systems, methods, apparatus, and computer device readable media that solve technical problems related to automated report generation including, in particular, abstracting relevant data and generating reports associated with hospital quality measures. In some implementations, queries to a large language model (LLM) are made using prompts that are selected to elicit responses that can generate hospital quality assessment reports. In some aspects, unstructured clinical data inputs to a large language model (LLM) can be augmented with structured data from patient health records prior to being queried with prompts. In this manner, the response to the prompts can be formulated using structured and unstructured patient health record data. In some examples, the patient data can be in the form of electronic health records. The electronic health records can be presented and stored in a well understood format.

In some examples, some or all of the disclosed functions or operations may be performed in a secure cloud server or a virtual private server. Performing operations in a secure cloud server or virtual private server can help ensure that sensitive patient records are secure and any relevant privacy regulations are maintained.

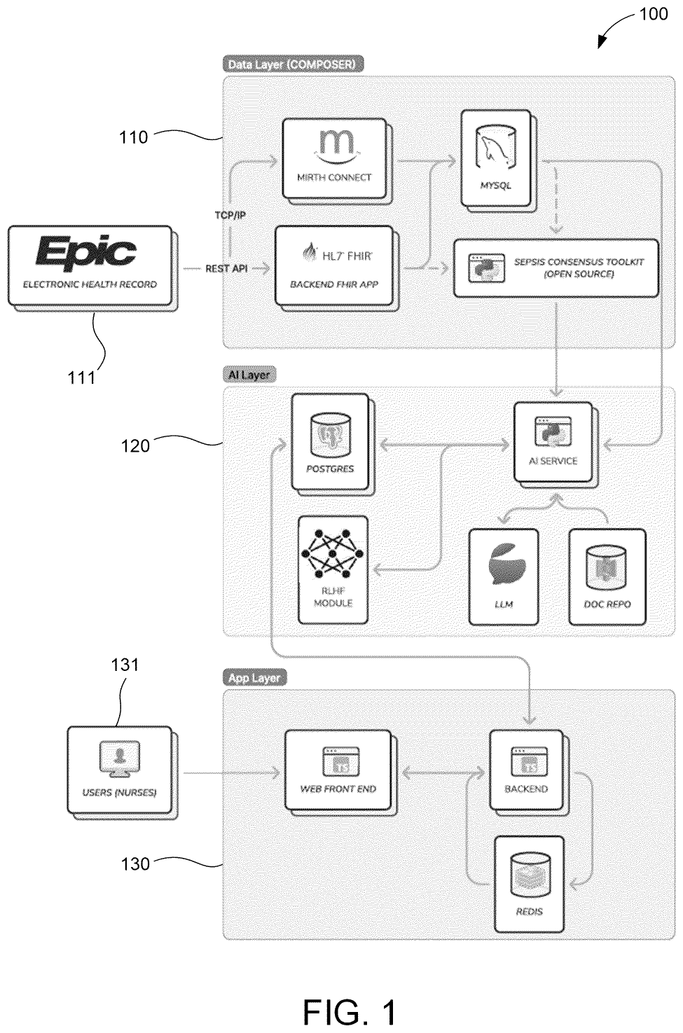

FIG. 1 illustrates a simplified flow diagram 100 describing a process flow associated with an automatic generation of assessment reports, using, for example, sepsis and septic shock quality criteria. In general, the flow diagram 100 may include three major divisions: a collection process 110, an inference process 120, and an output process 130.

The collection process 110 generally collects data associated with patient health records. For example, the collection process 110 can include receiving electronic health records 111 and also processing the electronic health records 111 in preparation for the inference process 120. The electronic health records 111 can include one or more patient health records, such as patient health records associated with a hospital department, a complete hospital, or a medical group. In some implementations, formatting of the electronic health records 111 can conform to one or more electronic health record (EHR) formats including, but not limited to EHR formats in compliance with Epic Systems, Fast Healthcare Interoperability Resources, or the like. The collection process 110 may include processes and/or procedures that can include processing by a REST API, a SQL server, or a SQL query.

The inference process 120 may include one or more processes associated with using artificial intelligence to generate a report based on the electronic health records 111. In some aspects, the inference process 120 can include processes associated with executing a trained neural network. For example, the inference process 120 may include executing a large language model (LLM) by presenting one or more prompts and receiving one or more responses to the one or more prompts. The prompts may be selected or designed to elicit responses to generate a report. In some examples, the prompts may be selected or designed to generate a hospital quality report. In some instances, the hospital quality report may be in accordance with the CMS Severe Sepsis and Septic Shock Management Bundle (and the report is sometimes referred to as a SEP-1 report or abstraction), a Central Line Associated Blood Stream Infections (CLABSI) measure, a Patient Safety Indicator 90 (PSI-90), or any user or agency specified quality measure. In some aspects, the inference process 120 may include performing a retrieval-augmented generation (RAG) process prior to or coincident with executing the LLM. The RAG process can combine structured and unstructured EHR data as inputs to the LLM to increase response accuracy.

For example, without RAG, the LLM takes the inputs (prompts, unstructured EHR data, etc.) and generates a response based on information available within its context length. With RAG, an information retrieval operation is included that utilizes the prompt to first pull precise information from additional data sources, in this case EHR information and quality measure guidelines. The prompt and the additional data are both given to the LLM. The LLM can use the additional data and the original LLM training data to create better responses.

The output process 130 can include processes that can include presenting or delivering quality abstraction reports generated by the inference process 120 to a user. In some examples, the output process 130 can format data from the inference process 120 into a recognizable (standardized) report. In addition, the output process 130 can elicit feedback from a user regarding the presented data, including, for example, a SEP-1 report. In some implementations, the output process 130 can use a variety of Web-based tools to present data and receive human feedback 131. In some implementations, the human feedback 131 may be stored in a human feedback record.

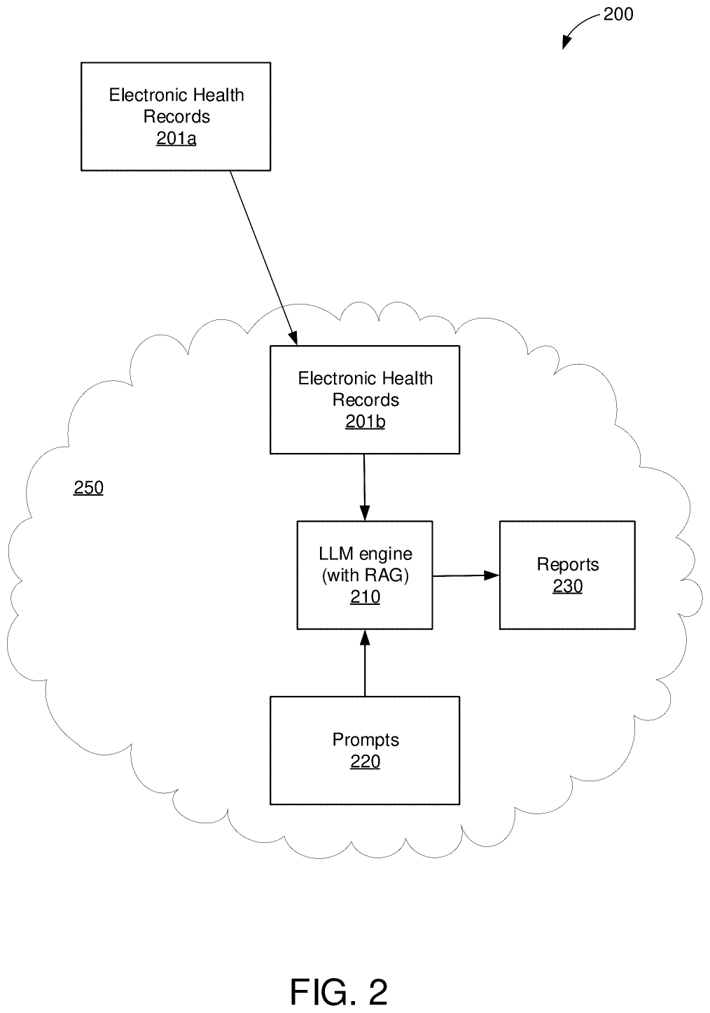

FIG. 2 shows an example simplified block diagram of a report generating system 200. In some examples, some or all of the report generating system 200 may be implemented on or executed within a virtual private cloud 250. The report generating system 200 can include an LLM engine 210. In some examples, the LLM engine 210 may be enhanced with a retrieval-augmented generation (RAG) processes. Operations performed by or associated with the report generating system 200 may provide a technical solution to a technical problem. Executing operations to query an LLM to generate quality abstraction reports is a technical problem rooted in computer technology. Analyzing patient health records for large numbers of patients can be arduous and time consuming. The technical solution provided by the report generating system 200 may provide a solution that is sufficiently efficient, particularly compared to manually reviewing hundreds or thousands of pages of patient health records.

As used herein, any “engine” may include one or more processors or a portion thereof. A portion of the one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specifics or other considerations, an engine can be centralized or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein, can be implemented locally with a processor or can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

The LLM engine 210 can be based on any feasible LLM. In some implementations, the LLM engine 210 may use open source or proprietary LLMs. As noted above, the LLM engine 210 may include a RAG process or engine that can update or augment LLM data with data from electronic health records. For example, electronic health records 201a (which can be examples of the electronic health records 111 of FIG. 1) may be transferred or uploaded to the virtual private cloud 250 as electronic health records 201b. In this manner, the LLM engine 210 may update or augment LLM data with the electronic health records 201b through a RAG process.

In some examples, the electronic health records 201b may be received as Fast Healthcare Interoperability Resources (FHIR) version R4, but other formats and versions are possible.

In some examples, data for patient medication administration may not be easily accessible through the electronic health records 201b. In some implementations, a proprietary application programming interface may be used to retrieve patient medication administration information from any feasible patient health record.

The report generating system 200 includes prompts 220 that are provided to the LLM engine 210. In response to the prompts 220, the LLM engine 210 can output portions of, or complete reports 230. The reports 230 can include sepsis and septic shock reports (data abstractions from the patient EHRs), CLABSI reports, or PSI-90 reports, among others. In some examples, the reports 230 can include data that fulfills reporting required by Centers for Medicare and Medicaid Services (CMS), NHSN, AHRQ, American Heart Association (AHA), or other payer groups. In some other examples, the reports 230 may include a completed SEP-1, CLABSI, or PSI-90 abstraction which may be presented to a user as a file or displayed through a web application (user interface). A SEP-1 abstraction may be a report defined by the CMS that includes data related to the diagnosis and treatment of Sepsis and Septic shock. In some examples, the prompts 220 may include or be derived from CMS guidelines to abstract data from health records to complete sepsis and septic shock assessments.

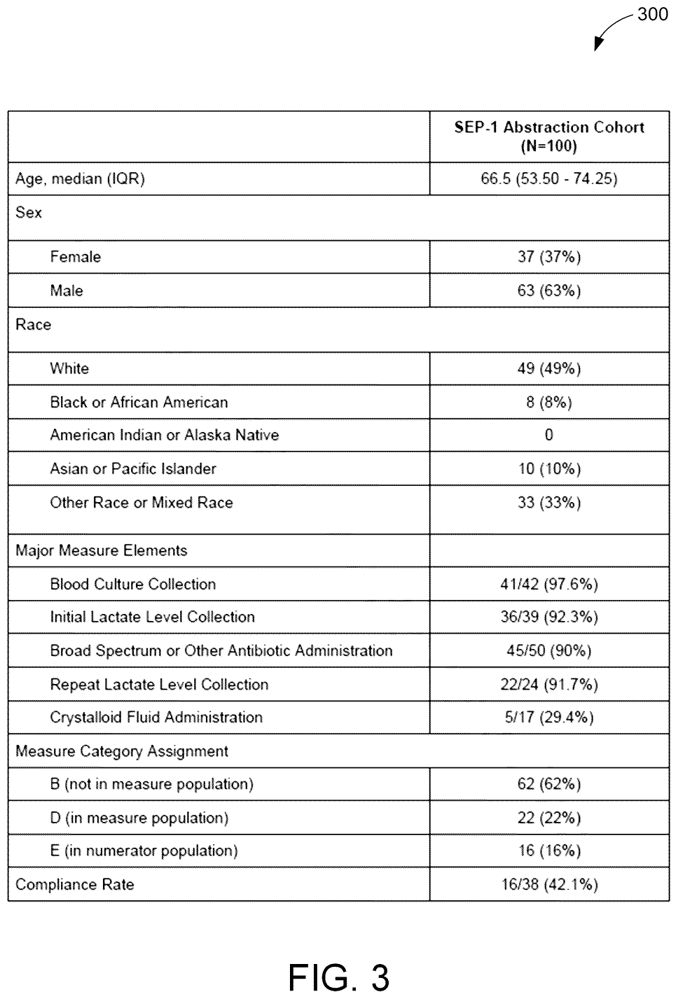

Implementations of the apparatus and/or systems depicted in FIGS. 1 and 2 have been tested to assess performance. In some examples, the apparatus and/or systems disclosed herein have been tested with a cohort of 100 cases representing five months of SEP-1 abstractions at two hospitals. FIG. 3 shows a table 300 outlining the demographics of the cohort. Outputs from the report generating system 200 were compared against outputs that were prepared manually through direct human preparation. Statistical comparisons were performed to determine how well the reports 230 match manually prepared reports.

One measurement comparing the LLM system (the report generating system 200) to the human abstractors and measure category agreement (pass, fail, or out-of-measure) has shown good results. Agreement was tested using Cohen's kappa with a two-sided test. Ten disagreements between the LLM system and human abstractors were adjudicated by a physician-expert and reported separately. In four of the ten disagreements, the LLM system was determined to be more accurate than human abstractors according to a physician-expert. Three independent trials of the LLM system were performed to evaluate consistency. Additionally, the difference between the predicted and reported compliance rate was tested using Pearson's chi-squared test. A P value less than 0.05 was interpreted as significant for all analyses. All statistical analyses were performed using Python version 3.11, the SciPy package version 1.10, and the stats model package version 0.13.5.

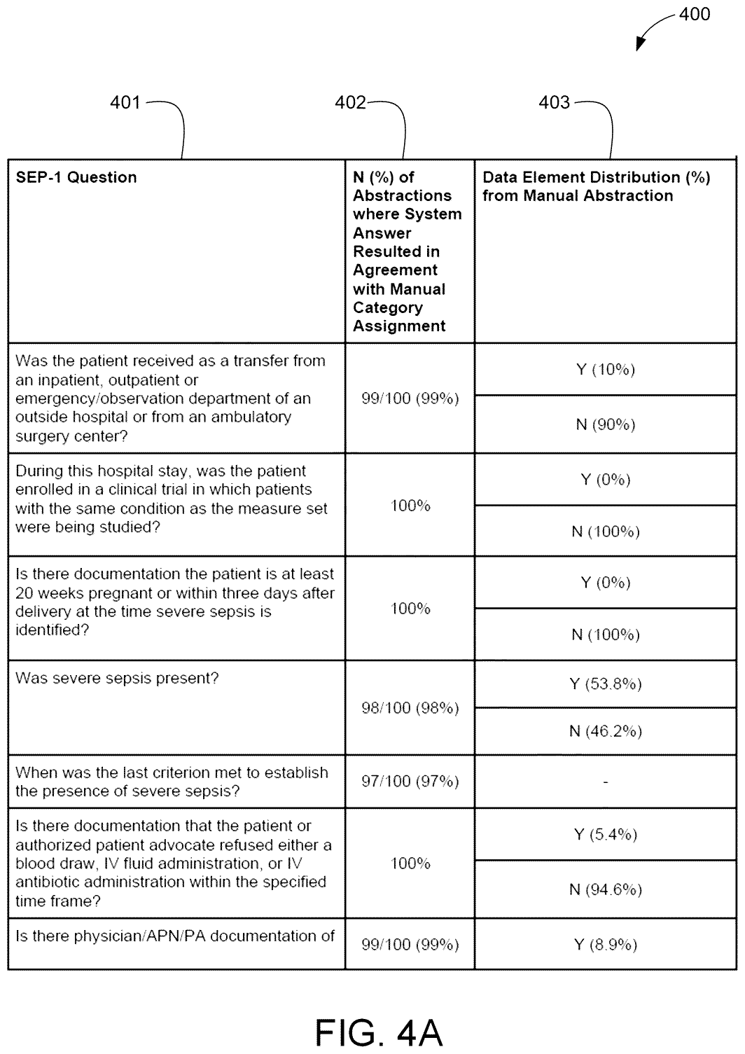

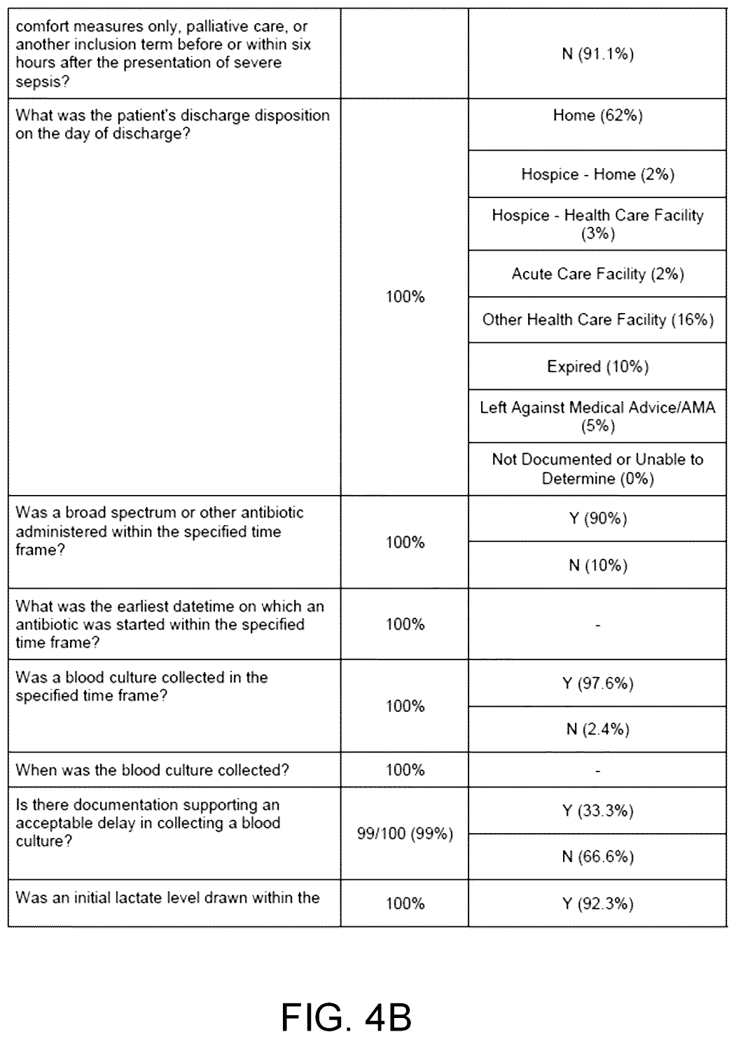

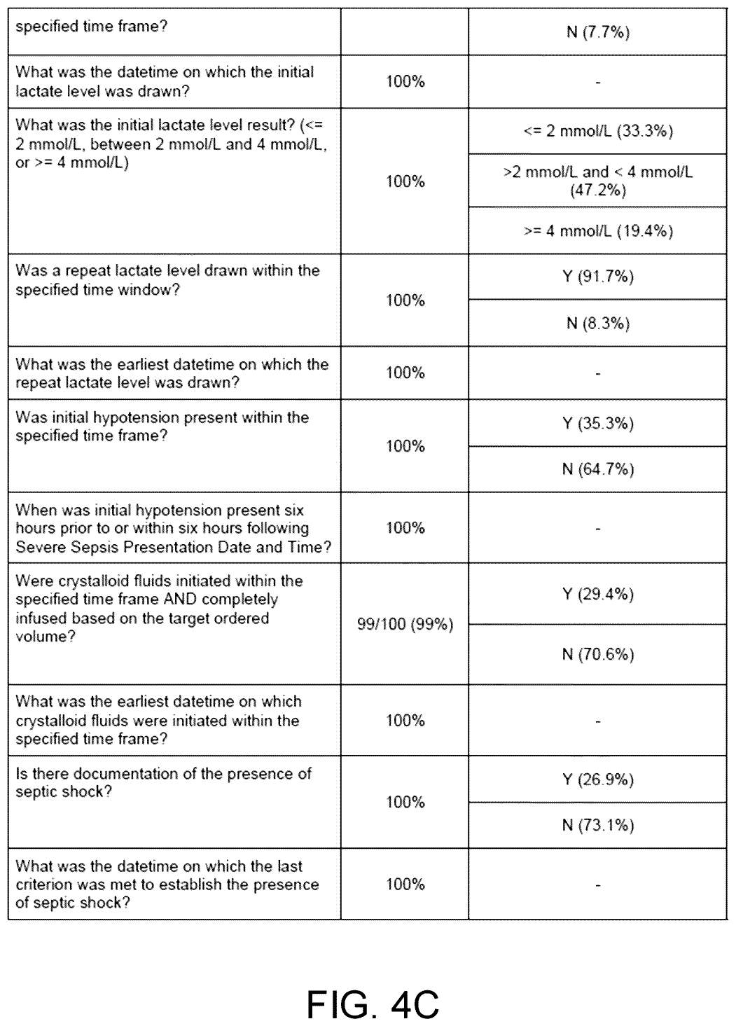

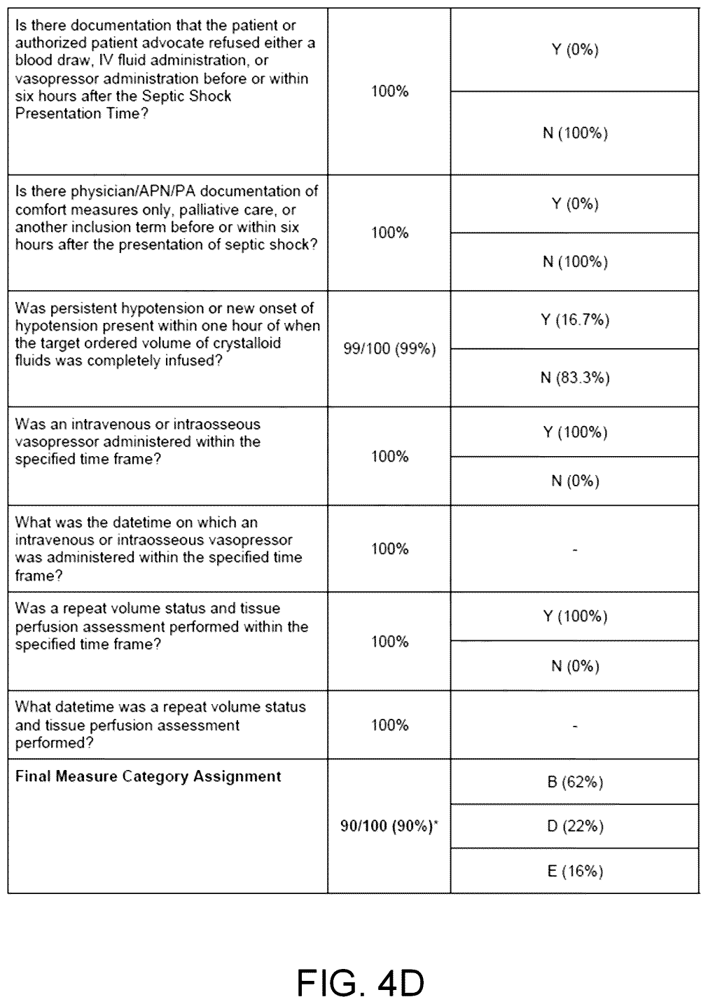

FIGS. 4A-4D show a table 400 that shows how well the LLM output agrees with (is consistent with) the same report prepared by human abstractors. Column 401 shows a question or category. Column 402 includes agreement between an output of the LLM system and an equivalent output produced by human abstractors. Column 403 shows a distribution of answers. Generally, table 400 shows that the LLM system (the report generating system 200) is well suited to produce an output that may be used to provide SEP-1 information.

In some implementations, the output of the report generating system 200 may be presented to a user, in some cases on a display of a computing device, the display of a mobile computing device, or any other feasible display. In some examples, the output of the report generating system 200 can be displayed through a web-based interface.

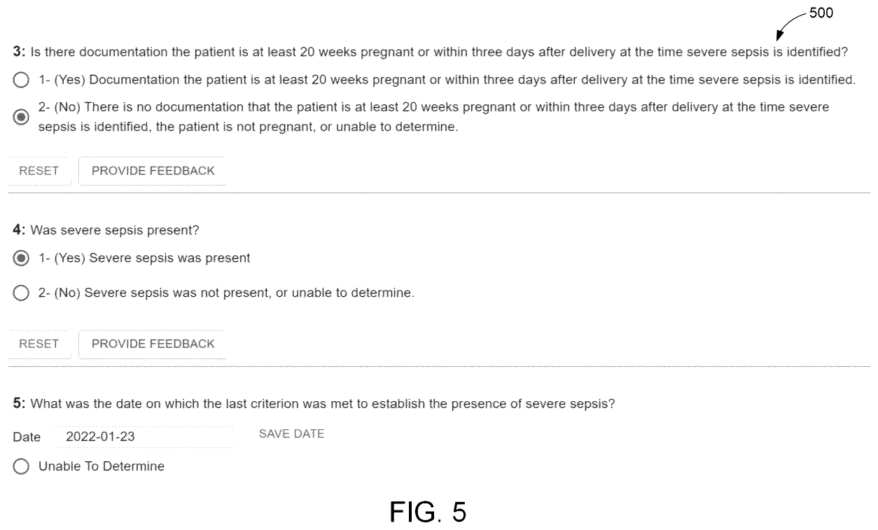

In some examples, a user can provide feedback in response to the output from the report generating system 200. FIG. 5 shows an example user interface 500 for providing feedback. In some examples, the user interface may include a web-based interface for receiving user inputs (feedback) regarding a report.

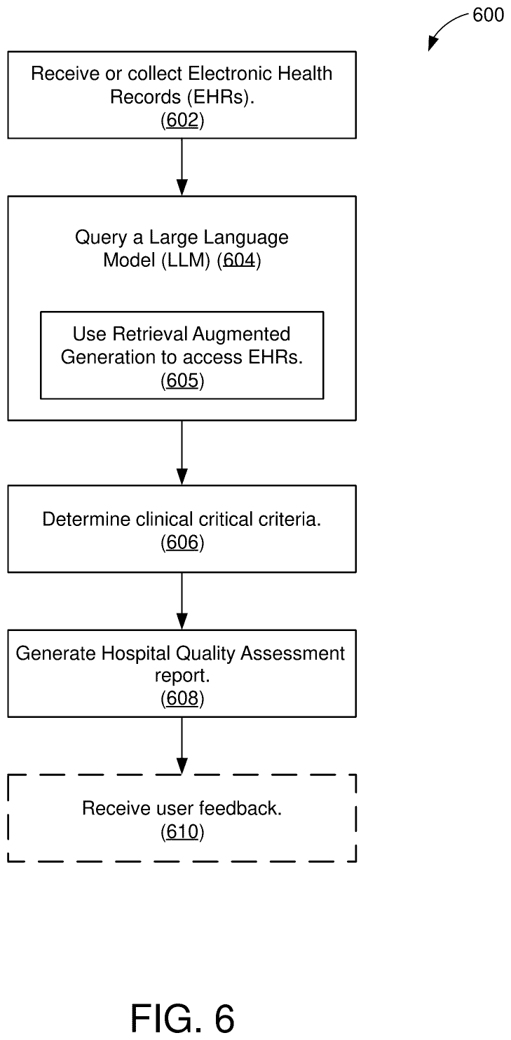

FIG. 6 is a flow chart showing an example method 600 for generating a hospital quality abstraction report. Some examples may perform the operations described herein with additional operations, fewer operations, operations in a different order, operations in parallel, and some operations differently. The method 600 is described below with respect to the report generating system 200 of FIG. 2, however, the method 600 may be performed by any other suitable system device, or apparatus. Operations associated with the method 600 may be performed on any suitable computing device. In some examples, the method 600 may be performed partially or entirely within a virtual private cloud to safeguard sensitive patient information.

The method 600 begins in block 602 as health records are received or collected by the report generating system 200. The health records may be associated with one or more patients. In some cases, the health records can be electronic health records. In other cases, the health records can be patient reported outcomes or data from data collection devices. In some examples, the health records may be associated with treatment received at a hospital or other treatment center within a particular time period. The health records may include static patient characteristics (height, weight), patient vital-signs, patient comorbidities, patient demographics, patient procedures, patient tests (blood tests, x-rays, vital signs), ordered medications, administered medications, clinical notes, signals from wearable devices, patient-reported audio or text, and the like.

Next, in block 604 an LLM is queried. In some aspects, the LLM may be queried with prompts associated with CMS or other guidelines for generating a report, such as a SEP-1 report. In some aspects, the prompts may be selected to elicit a response for each hospital quality measure element. The LLM can be any feasible LLM such as, but not limited to, the open-source general-purpose SOLAR 10.7B model with 8-bit quantization. In some examples, chain-of-thoughts and/or few-shot prompting strategies may also be used to elicit responses from the LLM.

In some aspects, LLM data may be enhanced with a RAG process in block 605. For example, the EHRs collected in block 602 may be used to augment data in the LLM through a RAG process. In this manner, queries to the LLM may return answers related to the EHRs.

Next, in block 606 clinical criteria is determined from the EHRs. For example, the report generating system 200 may use one or more utilities to determine or establish a presence of clinical criteria such as Systemic Inflammatory Response Syndrome (SIRS) criteria, Laboratory Confirmed Bloodstream Infection (LCBI) criteria, organ failure criteria, etc.

Next, in block 608 the report generating system 200 generates a hospital quality abstraction report. For example, the report generating system 200 can use the response to the prompts from the LLM (which may be enhanced with a RAG process to incorporate data from the EHRs in blocks 604 and 605) as well as clinical criteria (from block 606) to generate a hospital quality abstraction report. In some cases, the hospital quality abstraction report can include content to respond to each hospital quality measure element.

Next, in block 610, user feedback can be received. In some cases, the user feedback can be in response to elements contained within the hospital quality abstraction report. In some aspects, operations associated with block 610 can be optional, as shown with dashed lines in FIG. 6. In some examples, the user feedback may be received through a user interface that can present one or more aspects of the hospital quality abstraction report. The user interface can display dialog boxes, radio buttons, or the like to capture the user feedback. In some cases, the user feedback can be incorporated into the hospital quality abstraction report.

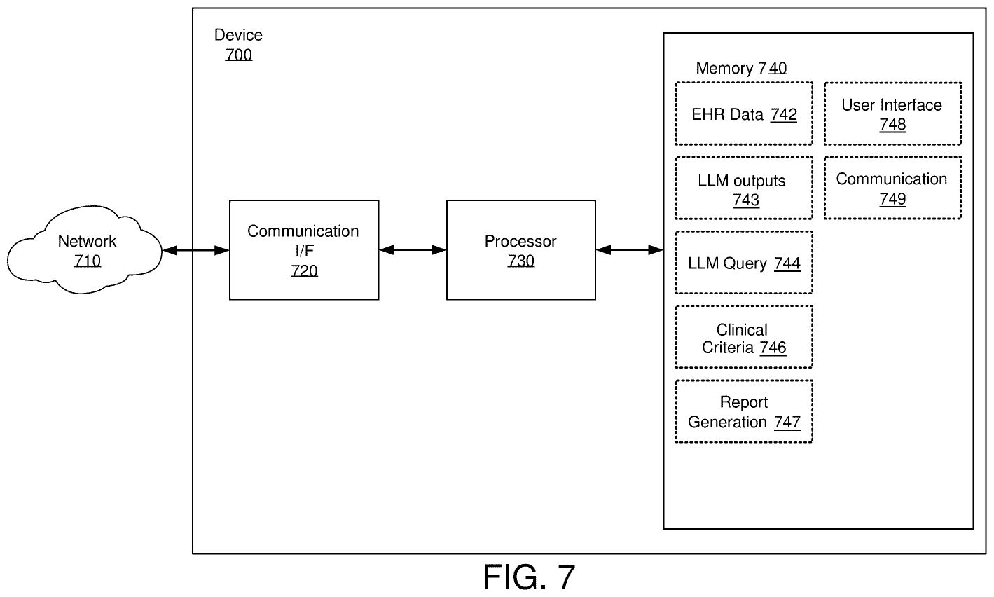

FIG. 7 shows a block diagram of a device 700 that may be one example of the report generating system 200 of FIG. 2. The device 700 may include a communication interface 720, a processor 730, and a memory 740. In some examples, the device 700 may be implemented within one or more secure, remote servers. For example, the device 700 may exist wholly or partially within a HIPAA-compliant virtual private cloud.

The communication interface 720, which may be coupled to a network (such as a network 710) and to the processor 730, may transmit signals to and receive signals from other wired or wireless devices, including remote (e.g., cloud-based) storage devices, cameras, processors, compute nodes, processing nodes, computers, mobile devices (e.g., cellular phones, tablet computers and the like) and/or displays. For example, the communication interface 720 may include wired (e.g., serial, ethernet, or the like) and/or wireless (Bluetooth, Wi-Fi, cellular, or the like) transceivers that may communicate with any other feasible device through any feasible network.

The processor 730, which is also coupled to the memory 740, may be any one or more suitable processors capable of executing scripts or instructions of one or more software programs stored in the device 700 (such as within memory 740).

The memory 740 may include EHR data 742 that may be locally stored patient EHRs. In some examples, the EHR data 742 can include health records stored in accordance with a Fast Healthcare Interoperability Resources format. The memory 740 can also include LLM data 743. The LLM data 743 can include data associated with any feasible LLM, including open source LLMs.

The memory 740 may also include a non-transitory computer-readable storage medium (e.g., one or more nonvolatile memory elements, such as EPROM, EEPROM, Flash memory, a hard drive, etc.) that may store the following software modules:
an LLM query module 744 to execute one or more queries with respect to an LLM;
a clinical criteria module 746 to determine clinical criteria;
a report generation module 747 to generate one or more hospital quality abstraction reports;
a user interface module 748 to display or publish one or more reports as well as receive feedback from a user; and
a communication module 749 to communicate with other devices.

Each software module includes program instructions that, when executed by the processor 730, may cause the device 700 to perform the corresponding function(s). Thus, the non-transitory computer-readable storage medium of memory 740 may include instructions for performing all or a portion of the operations described herein.

Execution of the LLM query module 744 can query the LLM data 743 with one or more prompts to receive responses that provide data for a hospital quality abstraction report. In some examples, the prompts may be selected to elicit responses for a severe sepsis and septic shock abstraction (SEP-1) required by Centers for Medicare and Medicaid Services (CMS). In some examples, the prompts may be selected to elicit responses for a CLABSI abstraction by NHSN. In some examples, the prompts may be selected to elicit responses for a PSI-90 abstraction by AHRQ. In some examples, the prompts may be selected to elicit responses for a custom abstraction by a hospital or payer. In some implementations, execution of the LLM query module 744 can also execute a RAG process to include EHR data 742 with the LLM data 743. In some examples, execution of the LLM query module 744 can cause the processor 730 to determine medication administration information from EHR data 742. In some cases, execution of the LLM query module 744 can determine medication administration information through a proprietary Application Programming Interface (API).

In some implementations, execution of the LLM query module 744 includes querying the LLM data 743 with prompts that are selected to elicit responses for hospital quality abstractions required by CMS or other payers or quality organizations. In some examples, the LLM query module 744 uses chain-of-thoughts and/or few-shot prompting strategies to elicit responses.

Execution of the clinical criteria module 746 can cause the processor 730 to search through the EHR data 742 looking for any information relevant to determining the clinical criteria. In some examples, execution of the clinical criteria module 746 can identify information relevant to Systemic Inflammatory Response Syndrome (SIRS) criteria and/or Laboratory Confirmed Bloodstream Infection (LCBI) criteria, or organ failure criteria.

Execution of the report generation module 747 can generate report data, including data for a sepsis and septic shock report or data for hospital quality abstractions required by CMS or other payers or quality organizations. Report data may be based on responses to queries from the LLM query module 744 and data from the clinical criteria module 746.

Execution of the user interface module 748 can display or publish report data, such as any data from the report generation module 747. In some examples, the user interface module 748 may display report data on a computer display or a display of a mobile computing device. In some other examples, the user interface module 748 may include a printer to print data from the report generation module 747. In some examples, execution of the user interface module 748 can cause the processor 730 to receive feedback from a user regarding information displayed on a user interface. For example, the user interface module 748 may display report data and receive user feedback in response to the report data.

The processor 730 may execute the communication module 749 to communicate with any other feasible devices. For example, execution of the communication module 749 may enable the device 700 to communicate via cellular networks conforming to any of the LTE standards promulgated by the 3^{rd} Generation Partnership Project (3GPP) working group, Wi-Fi networks conforming to any of the IEEE 802.11 standards, Bluetooth protocols put forth by the Bluetooth Special Interest Group (SIG), Ethernet protocols, or the like. In some embodiments, execution of the communication module 749 may enable the device 700 to communicate with a server to receive EHRs and/or LLMs. In some other embodiments, execution of the communication module 749 may implement encryption and/or decryption procedures.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations, or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise,” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components, or sub-steps.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method, the method comprising:
receiving, by a processor, one or more health records;
querying, by the processor, one or more prompts to a large language model (LLM) using a Retrieval Augmented Generation (RAG) process to query data from the one or more health records, additional corpora, and LLM outputs to determine a query response, wherein the RAG process further comprises querying electronic health record (EHR) data directly in Fast Healthcare Interoperability Resources (FHIR) format and extracting specific FHIR resources to incorporate structured EHR data as an additional context to answer quality abstraction questions,
further wherein an individual FHIR resource is flattened to create nodes within a knowledge graph structure representing an underlying clinical data ontology, and provided to the LLM a knowledge graph RAG (KG-RAG) to contextualize the LLM's understanding and ability to answer quality abstraction questions;
dynamically generating or modifying the one or more prompts based on an analysis of historical query responses, user feedback, and/or evolving clinical guidelines; and
generating, by a processor, a hospital quality abstraction report based on the query response.

2. The method of claim 1, further comprising using a second LLM to generate candidate enhanced prompts based on updated guidelines and feeding the candidate enhanced prompts and one or more few-shot examples based on user feedback to a Bayesian Optimization sub-routine to select an optimal prompt and a subset of the few-shot examples by maximizing an objective function comprising a match rate with user reported gold-standard labels.

3. The method of claim 2, wherein the user reported gold-standard labels comprise answers to quality measure questions.

4. The method of claim 1, wherein the querying includes one or more prompts based on guidelines for determining a hospital quality measure assessment from clinical records.

5. The method of claim 1, wherein the health records are stored in a predetermined format.

6. The method of claim 1, wherein the health records are compliant with a Fast Healthcare Interoperability Resources standard.

7. The method of claim 1, wherein the querying is performed within a Health Insurance Portability and Accountability Act (HIPAA) compliant virtual private cloud.

8. The method of claim 1, further comprising determining clinical criteria from the electronic health records, wherein the hospital quality abstraction report is based on the clinical criteria and the query response.

9. The method of claim 8, wherein the clinical criteria include any construct that may comprise of one or more clinical findings that are chained together via logical operators (such as AND, OR), such as Systemic Inflammatory Response Syndrome (SIRS), sequential organ failure assessment score (SOFA), Laboratory Confirmed Bloodstream Infection (LCBI) criteria, among others.

10. The method of claim 1, further comprising: receiving feedback from a user regarding the hospital quality abstraction report; and creating a feedback record based on the received feedback.

11. The method of claim 1, wherein the querying includes presenting one or more prompts following a chain-of-thoughts prompting strategy.

12. The method of claim 1, wherein the querying includes presenting one or more prompts following a few-shot prompting strategy.

13. The method of claim 1, wherein the querying includes presenting one or more prompts selected to elicit responses that generate hospital quality assessment report data.

14. A method, the method comprising:
receiving, by a processor, one or more health records;
querying, by the processor, one or more prompts to a large language model (LLM) using a Retrieval Augmented Generation (RAG) process to query data from the one or more health records, additional corpora, and LLM outputs to determine a query response, wherein the RAG process further comprises querying electronic health record (EHR) data directly in Fast Healthcare Interoperability Resources (FHIR) format and extracting specific FHIR resources to incorporate structured EHR data as an additional context to answer quality abstraction questions,
further wherein an individual FHIR resource is flattened to create nodes within a knowledge graph structure representing an underlying clinical data ontology, and provided to the LLM a knowledge graph RAG (KG-RAG) to contextualize the LLM's understanding and ability to answer quality abstraction questions;
wherein the one or more prompts is generated using a second LLM to generate candidate prompts based on updated guidelines and feeding the candidate prompts and one or more few-shot examples based on user feedback to a Bayesian Optimization sub-routine to select an optimal prompt and a subset of the few-shot examples by maximizing an objective function comprising a match rate with user reported gold-standard labels, wherein the user reported gold-standard labels comprise answers to quality measure questions;
dynamically generating or modifying the one or more prompts based on an analysis of historical query responses, user feedback, and/or evolving clinical guidelines;
generating, by a processor, a hospital quality abstraction report based on the query response; and
outputting the hospital quality abstraction report.

15. A non-transitory computer-readable storage medium comprising instructions that, when executed by one or more processors of a device, cause the device to perform operations comprising:
receiving, by a processor, one or more health records;
querying, by the processor, one or more prompts to a large language model (LLM) using a Retrieval Augmented Generation (RAG) process to query data from the one or more health records, additional corpora, and LLM outputs to determine a query response, wherein the RAG process further comprises querying electronic health record (EHR) data directly in Fast Healthcare Interoperability Resources (FHIR) format and extracting specific FHIR resources to incorporate structured EHR data as an additional context to answer quality abstraction questions,
further wherein an individual FHIR resource is flattened to create nodes within a knowledge graph structure representing an underlying clinical data ontology, and provided to the LLM a knowledge graph RAG (KG-RAG) to contextualize the LLM's understanding and ability to answer quality abstraction questions;
dynamically generating or modifying the one or more prompts based on an analysis of historical query responses, user feedback, and/or evolving clinical guidelines; and
output, from the processor, a hospital quality abstraction report based on the query response.

16. An apparatus comprising:
a processor configured to:
receive one or more health records;
query one or more prompts to a large language model (LLM) using a Retrieval Augmented Generation (RAG) process to query data from the one or more health records, additional corpora, and LLM outputs to determine a query response, wherein the RAG process further comprises querying electronic health record (EHR) data directly in Fast Healthcare Interoperability Resources (FHIR) format and extracting specific FHIR resources to incorporate structured EHR data as an additional context to answer quality abstraction questions,
further wherein an individual FHIR resource is flattened to create nodes within a knowledge graph structure representing an underlying clinical data ontology, and provided to the LLM a knowledge graph RAG (KG-RAG) to contextualize the LLM's understanding and ability to answer quality abstraction questions;
dynamically generate or modify the one or more prompts based on an analysis of historical query responses, user feedback, and/or evolving clinical guidelines; and
generating, by a processor, a hospital quality abstraction report based on the query response.
